(19) United States
(12) Patent Application Publication
(10) Pub. No.: US 2026/0357186 A1
(43) Pub. Date: Nov. 12, 2026

(54) DEVICE FOR HANDLING CELL CULTURES

(71) Applicant: Universität Linz, Linz (AT)

(72) Inventors: Georg Csukovich, Wien (AT); Marlene Rezk, Herzogsdorf (AT)

(21) Appl. No.: 19/132,720

(22) PCT Filed: Nov. 22, 2023
(86) PCT No.: PCT/EP2023/082691

§ 371 Date: May 23, 2025

(30) Foreign Application Priority Data
Nov. 25, 2022 (EP) 22209586.1

(51) Int. Cl. C12M 1/00; C12M 3/00

(52) U.S. Cl. CPC C12M 41/14; C12M 23/40; C12M 23/48; C12M 29/00

(57) ABSTRACT

A device for treating cell cultures comprises an incubator receiving the cell cultures that can be connected by conveying lines (17, 18) to a supply device (2) supplying a medium to the cell cultures and to a disposal device (3) receiving the medium pumped out of the cell cultures. The incubator (1) comprises a stack (5) of cell culture containers (4) with container accesses (6) aligned parallel on one stack side and a cross slide (7) on the access side of the container stack (5). The cross slide (7) carries an actuating head (15) that can be moved parallel to align the container accesses (6) and has a cannula (16) that can be inserted into the container accesses (6) by moving the actuating head (15), which can be connected either to the conveying line (17; 18) of the supply device (2) or to the disposal device (3). The conveying lines (17, 18) have a superhydrophobic inner surface, in which the contact angle of the liquid droplets is greater than 150°.

### TECHNICAL FIELD

The invention relates to a device for treating cell cultures, comprising an incubator for receiving the cell cultures, which incubator can be connected by conveying lines on the one hand to a supply device for supplying a medium to the cell cultures and on the other hand to a disposal device for receiving the medium pumped out of the cell cultures.

### DESCRIPTION OF THE PRIOR ART

For cultivation, cell cultures must be supplied with nutrients and additives, which are added to the cell cultures in the form of liquid media. Since the cell cultures must be supplied with fresh, nutrient-rich media once the nutrients have been consumed, media changes involve a considerable amount of repetitive work, because the cell culture containers containing the cell cultures must be removed from the incubator in order to remove the consumed media from the cell culture containers and fill them with fresh media, under the difficult conditions of the required sterility. To avoid this work, it has already been proposed (EP 0 447 256 A2) to connect the incubator to a supply device for supplying the cell cultures with a medium on the one hand and to a disposal device for the medium removed from the cell cultures on the other. The supply device comprises a storage container for fresh medium, which is supplied by means of a conveying pump through a conveying line to the cell culture container inside the incubator. To remove the consumed medium, the cell culture container is connected via a conveying line to the disposal device, which has a collection container for consumed medium, which in turn is removed from the cell culture container by means of a pump. To prevent contamination of the cell cultures, the supply device on the outlet side, the disposal device on the inlet side and the incubator on the inlet and outlet sides are equipped with sterilization filters. Apart from the construction costs incurred by the sterilization filters, another disadvantage is that devices of this type are not suitable for the simultaneous treatment of several cell cultures in a common incubator.

In order to treat several cell cultures in a common incubator, it is known (EP 2 821 477 A1) to provide the cell cultures in Petri dishes which are distributed on two coaxial, independently drivable turntables and can be supplied with a corresponding culture medium via an axis-parallel adjustable pipette. A separate pipette is provided for extracting the consumed culture medium. Apart from the fact that the constructively complex device is only suitable for use with Petri dishes, the question of how to ensure the necessary sterility remains open.

In an automatic pipetting and analysis device, it is known (DE 10 2004 057 450 A1) to provide two cross slides arranged at a distance above each other, one for a sample plate and the other for a pipetting head, in order to supply the samples in the sample plate with corresponding solutions when the pipetting head is aligned with the sample plate by means of its cross slide. However, by means of the cross slide for the pipetting head, the sample plate can also be moved from an analysis area into an incubator or from the incubator into the analysis area after coupling with the cross slide for the sample plate.

In order to be able to detect the local position of individual cell culture containers or the local position of handling units consisting of several cell culture containers forming a stack in an incubator after each movement, it has already been proposed (EP 4 036 206 A1, EP 4 036 212 A1) to optically track the individual handling units from a cell culture container or from a stack thereof so that the respective final position of the individual handling units, each provided with an identifier, can be determined from the recorded image data. The camera used for this purpose can be stationary mounted (EP 4 036 206 A1) but can also be moved by means of a robot arm (EP 4 036 212 A1).

In order to avoid the handling disadvantages associated with the use of cell culture containers in particular, it is also known (US 2021/269761 A1) to provide an element, preferably formed as a microscope carrier, which comprises a cultivation channel for cultivating the cells, a storage channel for receiving the cultivated cells and a transfer channel connecting the cultivation channel to the storage channel. This element is connected to a storage container for an initial cell suspension and a storage container for a cell culture medium in order to first fill the cultivation channel with the initial cell suspension before a cell culture medium is pumped into the cultivation channel. After cell cultivation, the cell culture is transferred to the storage channel for further use. Since the cells of the initial cell suspension should grow in the cultivation channel, the cultivation channel can preferably be provided with a hydrophilic coating that promotes cell growth. However, the cells should not grow in the transfer channel or in the storage channel if possible. For this reason, the walls of these channels can be provided with a hydrophobic coating. Due to the closed system consisting of the element connected to the storage container, it is relatively easy to operate the system under sterile conditions, especially since the system does not require pipette cannulas that can be inserted into cell culture containers.

### DESCRIPTION OF THE INVENTION

The invention is based on the problem of providing a device for the simultaneous treatment of several cell cultures in separate cell culture containers using a common incubator in such a way that a sterile media exchange in the individual cell culture containers can be ensured by means of comparatively simple means of construction.

Based on a device of the type described above, the invention solves the problem thereby that the incubator comprises a stack of cell culture containers with container accesses aligned parallel on one stack side and a cross slide on the access side of the container stack, the cross slide carries an actuating head that can be moved parallel to align the container accesses and has a cannula that can be inserted into the container accesses by moving the actuating head, which can be connected either to the conveying line of the supply device or to the disposal device, and that the conveying lines have a superhydrophobic inner surface, in which the contact angle of the liquid droplets is greater than 150°.

By arranging a stack of aligned cell culture containers and an actuating head that can be aligned opposite these cell culture containers by means of a cross slide in a common incubator, it is initially possible, using relatively simple means, to insert the cannula associated with the actuating head into the selected cell culture container through the container access when the actuating head aligned with a selected cell culture container is moved accordingly, as a prerequisite for automated medium exchange. For medium exchange, the cannula inserted into the cell culture container must first be connected to the conveying line for the disposal device so that the medium can be pumped out of the cell culture container and conveyed to the disposal device before the cannula is connected to the conveying line of the supply device and fresh medium is pumped from the supply line into the cell culture container. When the cannula is removed from the container access, the medium exchange for the selected cell culture container is complete and the device is ready for a medium exchange in another cell culture container.

Since the conveying lines between the supply device and the disposal device on the one hand and the cannula of the actuating head on the other hand have a superhydrophobic inner surface, no microbes can deposit in these conveying lines.

Under the normally required condition of a sterile supply device in the area of the parts that convey the medium, this means that a sterile supply and removal of the medium, which is largely free of contamination, can be assumed, so that additional sterilization filters are not necessary.

The decisive factor for the effect of the superhydrophobic inner surface of the conveying lines on the sterility of the device is that the contact angle of liquid droplets is greater than 150°. These contact angles result in a microscopic surface structure that allows air pockets to be trapped in the nanometer range. These air pockets cannot be displaced by surrounding liquid droplets, so that the surface is not wetted. Since microorganisms are present in the liquid phase, their deposition in the conveying lines is prevented, eliminating the need for additional sterilization measures.

In order to be able to adjust the alignment of the actuating head relative to the container accesses for different cell culture containers, the cross slide may have a displacement guide for the actuating head, which is mounted pivotably about an axis parallel to the base and perpendicular to the axis of the cannula.

Since, according to the invention, the incubator basically only needs to be equipped with a cross slide, and this cross slide can be easily combined with the actuating head to form a single unit, conventional incubators can be converted in accordance with the invention without any special construction work.

If, in this context, the conveying lines are connected to conveying pumps outside the incubator, the unit can not only be made more compact, but can also be advantageously limited to those components which are indispensable within the incubator for conveying the medium into and out of the cell culture containers.

### BRIEF DESCRIPTION OF THE INVENTION

The drawing shows an example of the invention.

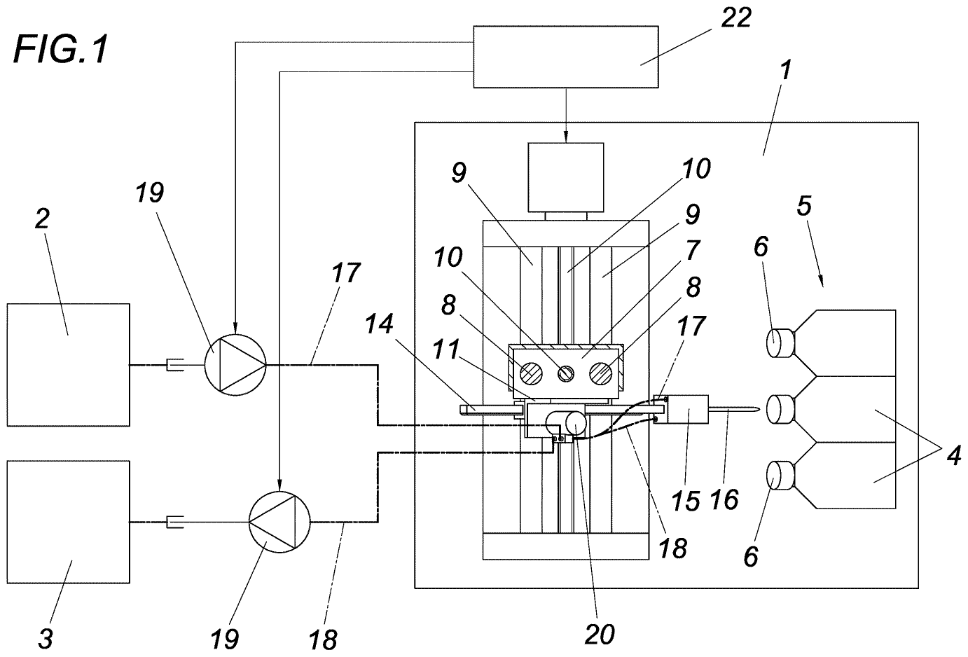

FIG. 1 shows a device according to the invention for treating cell cultures in a schematic block diagram with an incubator shown in horizontal section, and

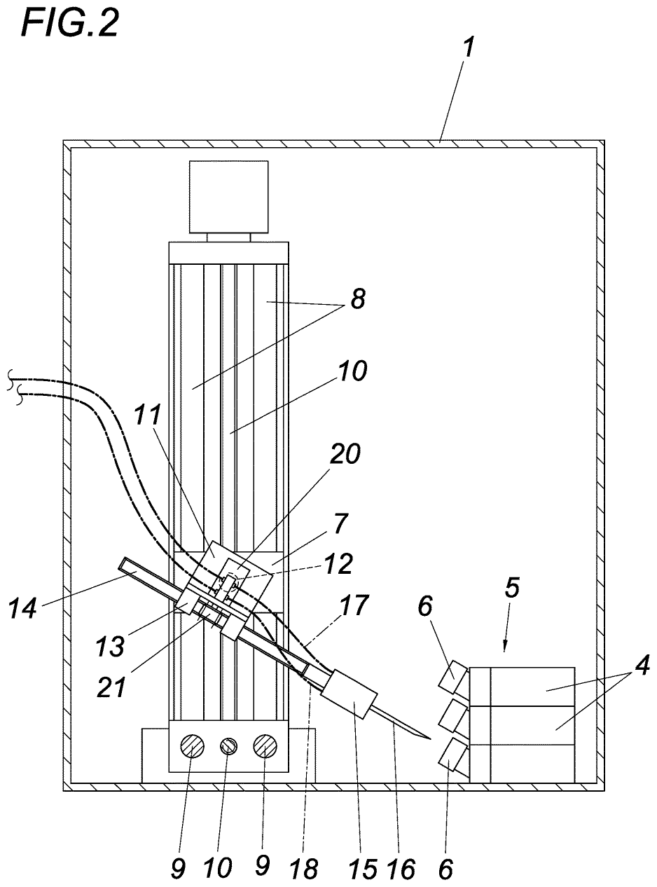

FIG. 2 shows the incubator according to FIG. 1 in an elevation view on a larger scale.

### WAYS OF EMBODYING THE INVENTION

The device for treating cell cultures according to FIG. 1 comprises an incubator 1, a supply device 2 for supplying the cell cultures with a medium containing nutrients and additives, which is usually liquid, and a disposal device 3 for receiving the medium pumped out of the cell cultures. For this purpose, the supply device 2 has a sterile storage container for the medium, which is removed from the cell cultures after consumption and conveyed to a collection container of the disposal device 3.

In incubator 1, several cell cultures are treated simultaneously in separate cell culture containers 4, usually cell culture bottles, which are aligned in parallel to form a stack 5 in a predetermined position on one side of incubator 1. The cell culture containers 4 are aligned so that their conventionally closure-provided container accesses 6 face the interior of the incubator 1 in a parallel position. On the side of the incubator 1 opposite the stack 5, a cross slide 7 is provided, which can be moved in height along a guide 8, which in turn is mounted on a guide 9 parallel to the base. Spindle drives 10 along the guides 8 and 9 are indicated as drive elements for the cross slide 7, but this is by no means mandatory.

A pivot bearing 11 with a pivot axis 12 parallel to the base-parallel guide 9 is provided on the cross slide 7, which carries a displacement guide 13 for an arm 14. An actuating head 15 with a cannula 16 extending in the direction of the arm 14 is provided at the end of the arm 14 facing the stack 5, which can be connected selectively to one of two conveying lines 17, 18, for example via a switching valve.

While the conveying line 17 connects the cannula 16 to the supply device 2 via a conveying pump 19, the conveying line 18 serves to connect the cannula 16 to the disposal device 3, the conveying line 18 also being provided with a conveying pump 19.

The cannula 16 can be aligned with a selected cell culture container 4 of the stack 5 using the cross slide 7 and the pivot bearing of the arm 14 and inserted into the cell culture container 4 by moving the arm 14 longitudinally through the respective container access 6, in order to either supply medium from the supply device 2 or pump consumed medium from the cell culture container 4 into the disposal device 3, depending on the connection of the cannula 16 to the conveying line 17 or 18. An actuating drive is provided for moving the actuating head 15, which, according to the embodiment, is formed as a rack and pinion drive with the arm 14 as a rack which meshes with a pinion 21 driven by a motor 20. However, other actuating drives are of course possible.

By means of a control device 22, which, depending on predefinable parameters, controls not only the actuating drives for the cross slide 7 and the actuating head 15, but also the conveying pumps 19 and any switching device for connecting the cannula 16 to the two conveying lines 17, 18, the process of medium exchange for the individual cell cultures can be largely automated in a relatively simple manner. The replacement of the storage container of the supply device 2 and the collection container of the disposal device for the consumed medium, which is necessary depending on the consumption of the medium, also does not require any special handling effort, because only the conveying lines 17, 18 need to be connected in a sterile manner to the supply device 2 and to the disposal device 3 by means of appropriate line couplings 23. Due to the formation of the conveying lines 17, 18 with a superhydrophobic inner surface, sterilization filters that would otherwise be necessary are not required, thus eliminating the construction and maintenance costs associated with these filters. A superhydrophobic surface is characterized by a contact angle of the liquid droplets greater than 150°. Such surfaces can be formed by laser-modified metal surfaces, but for this embodiment, polymer surfaces modified with apolar groups, such as siloxanes with long apolar side chains, are preferred.

1. A device for treating cell cultures, said device comprising:
an incubator receiving the cell cultures, said incubator being configured to be connected by conveying lines to a supply device supplying a medium to the cell cultures and to a disposal device receiving the medium pumped out of the cell cultures;
wherein the incubator comprises a stack of cell culture containers with container accesses aligned parallel on an access side of the stack of the containers, and a cross slide on the access side of the stack of the containers;
wherein the cross slide carries an actuating head that is supported so for movement in parallel so as to align with the container accesses, and the cross slide has a cannula configured for insertion into the container accesses by moving the actuating head, said cannula being selectively connectable either to the conveying line of the supply device or to the disposal device, and
wherein the conveying lines have a superhydrophobic inner surface, in which the contact angle of the liquid droplets is greater than 150°.

2. The device according to claim 1, wherein the cross slide has a displacement guide operatively associated with the actuating head, said displacement head being mounted pivotably about an axis parallel to a base of the device and perpendicular to an axis of the cannula.

3. The device according to claim 1, wherein the conveying lines are connected to conveying pumps outside the incubator.

4. The device according to claim 2, wherein the conveying lines are connected to conveying pumps outside the incubator.
